(19) United States
(12) Patent Application Publication
(10) Pub. No.: US 2024/0111998 A1
(43) Pub. Date: Apr. 4, 2024

(54) INFERRING DEVICE, INFERRING METHOD, AND TRAINING DEVICE

(71) Applicant: Preferred Networks, Inc., Tokyo-to (JP)

(72) Inventors: Daisuke MOTOKI, Tokyo-to (JP); Chikashi SHINAGAWA, Tokyo-to (JP); So TAKAMOTO, Tokyo-to (JP); Hiroki IRIGUCHI, Chiyoda-ku (JP)

(73) Assignee: Preferred Networks, Inc., Tokyo-to (JP)

(21) Appl. No.: 18/534,252

(22) Filed: Dec. 8, 2023

Related U.S. Application Data
(63) Continuation of application No. PCT/JP2022/023520, filed on Jun. 10, 2022.

(30) Foreign Application Priority Data
Jun. 11, 2021 (JP) 2021-098325

(51) Int. Cl. G06N 3/045; G06N 3/084

(52) U.S. Cl. CPC G06N 3/045; G06N 3/084

(57) ABSTRACT

An inferring device includes one or more memories; and one or more processors. The one or more processors are configured to input information on each atom in an atomic system into a second model to infer a difference between energy based on a first-principles calculation corresponding to the atomic system and energy of an interatomic potential function corresponding to the atomic system.

### CROSS REFERENCE TO RELATED APPLICATIONS

This application is continuation application of International Application No. JP2022/023520, filed on Jun. 10, 2022, which claims priority to Japanese Patent Application No. 2021-098325, filed on Jun. 11, 2021, the entire contents of which are incorporated herein by reference.

### FIELD

This disclosure relates to an inferring device, an inferring method, and a training device.

### BACKGROUND

When generating the interatomic potential, a 2-body potential function is added inside a potential function and a curve of the 2-body potential is used together. This is for generating the interatomic potential with high reproducibility by adding correction terms to the 2-body potential from the physical consideration.

For example, the interatomic potential used for the existing MD (Molecular Dynamics) simulation often includes the 2-body potential function. An approach of training a neural network model to obtain NNP (Neural Network Potential) being the interatomic potential is made, but an approach of using the 2-body potential is not made in this method.

### BRIEF DESCRIPTION OF DRAWINGS

FIG. 1 is a block diagram illustrating a training device according to an embodiment.

FIG. 2 is a flowchart illustrating processing by a training device according to an embodiment.

FIG. 3 is a diagram schematically illustrating a flow of data in data generation, model generation, and deduction according to an embodiment.

FIG. 4 is a graph of deducing a 2-body potential in NNP generated in a device according to an embodiment

FIG. 5 is a block diagram illustrating a training device according to an embodiment.

FIG. 6 is a flowchart illustrating processing of a training device according to an embodiment.

FIG. 7 is a block diagram illustrating an inferring device according to an embodiment.

FIG. 8 is a flowchart illustrating processing by an inferring device according to an embodiment.

FIG. 9 is a diagram schematically illustrating a flow of data in data generation, model generation, and deduction according to an embodiment.

FIG. 10 is a diagram illustrating one example of an implementation of an information processing system/device according to an embodiment.

### DETAILED DESCRIPTION OF THE INVENTION

According to one embodiment, an inferring device includes one or more memories; and one or more processors. The one or more processors are configured to input information on each atom in an atomic system into a second model to infer a difference between energy based on a first-principles calculation corresponding to the atomic system and energy of an interatomic potential function corresponding to the atomic system.

Hereinafter, embodiments of the present invention will be explained with reference to the drawings. The drawings and the explanation of the embodiments are indicated as examples and are not intended to limit the present invention.

First of all, some terms in this disclosure will be explained.

Interatomic potential (interaction potential energy between atoms) is a function of finding energy from the arrangement of atoms. This function is generally an artificial function. This is a function corresponding to a governing equation for MD (molecular dynamics) simulation. A non-limiting example of the interatomic potential is the Lennard Jones Potential.

In this description, the NNP (Neural Network Potential) approximates and expresses a potential function of an atomic system by a neural network. The neural network may be, for example, the GNN (Graph Neural Network) using, for example, a graph. The NNP in this description (model NN) is a trained neural network having an input layer into which information on each of atoms in an atomic system whose energy is to be inferred, a hidden layer which performs calculation based on the input information, and an output layer which outputs the energy of the atomic system.

The training of the neural network is performed using a training data set. The training data set includes, for each of a plurality of atomic systems, information on each of the atoms of the atomic system and a correct value of the energy of the atomic system. The correct value of the energy of the atomic system is a value of energy calculated by the first-principles calculation (for example, a calculation based on the DFT (density functional theory), a calculation based on the HF (Htree-Fock) method, a calculation based on the MP (Moeller-Plesset) method, or the like) for the atomic system. The training of the neural network calculates an error between an inferred value of the energy of the atomic system output by inputting the information on each of the atoms of the atomic system into the neural network and its correct value, for each of the atomic systems included in the training data set, and updates a weight parameter of the neural network by the error backpropagation method based on the error.

The NNP may output, in addition to the input energy of the atomic system, input secondary information such as charge of each of the atoms of the atomic system. In this case, the training data set of the neural network includes the correct value related to the charge, and the neural network is trained by the error backpropagation method based on the energy error and the error related to the charge. The NNP may have a function of calculating a differential value of the inferred energy with respect to the position of the atom, as a force applied to the atom. Further, the NNP may output input force applied to each of the atoms on the atomic system. In this case, the training data set of the neural network includes a correct value related to the force applied to each of the atoms, and the neural network is trained by the error backpropagation method based on the energy error and/or the error related to the charge, and the error related to the force applied to each of the atoms.

The information on the atom input into the model NN used for the NNP is, for example, information including the information on the type and position of each of the atoms. In this description, the information on the atom is called information related to the atom in some cases. Examples of the information on the position of the atom include information directly indicating the position of the atom by coordinates, information directly or indirectly indicating the relative positions between atoms, and so on. The information is expressed, for example, by the distance, the angle, the dihedral angle, or the like between atoms. For example, by calculating the information on the distance between two atoms or the angle among three atoms from the information on the coordinates of atoms and using them for input into the NNP as the information on the positions of the atoms, it is possible to secure invariance for the rotation and translation and enhance the accuracy of the NNP. For example, the information on the atom may be information directly indicating the position or information calculated from the positional information. Further, the information on the atom may include information related to charge and information related to binding in addition to the information on the type and the position of the atom.

In this description, the model NN outputs, for example, information related to energy. Examples of the information related to energy include energy, and examples of the information calculated based on information energy calculated based on the energy include force of each atom, stress (stress of the whole system), virial of each atom, virial of the whole system, and so on. The NNP may output information which can be calculated using the NNP such as charge of each atom, in addition to the information related to the energy.

A 2-body potential curve shows the relation between the distance and the energy of two atoms in the case where only the two atoms exist in the system.

A 2-body potential function is intended to express the whole energy by a sum of the 2-body potential curve as a sign of the potential. Generally, it is difficult to accurately reproduce the energy value or the like only from the 2-body potential function. The aforementioned Lennard Jones Potential is also this 2-body potential function.

### First Embodiment

In this embodiment, the information related to the 2-body potential is used as the training data together with data on a compound such as a molecule or crystal in training of the neural network model realizing the NNP.

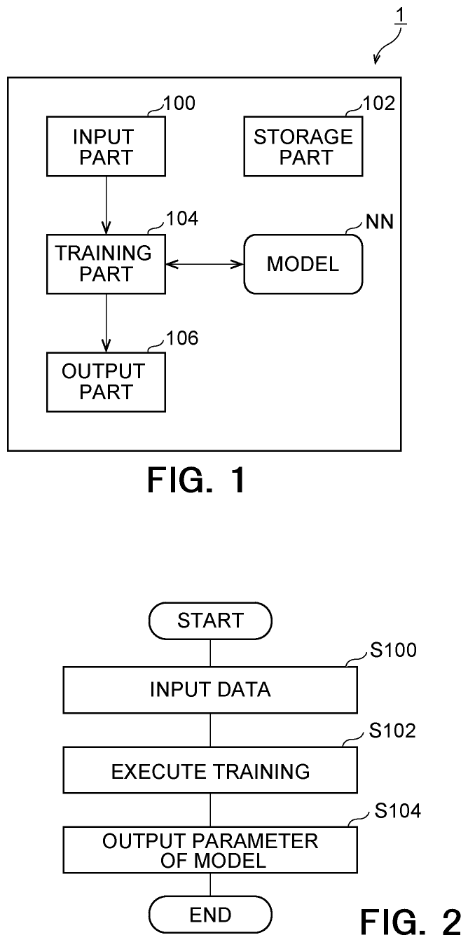

FIG. 1 is a block diagram illustrating an example of a training device according to this embodiment. A training device 1 includes an input part 100, a storage part 102, a training part 104, and an output part 106. The training device 1 is a device that trains the model NN based on input data by an arbitrary appropriate machine learning technique.

The input part 100 accepts input of data in the training device 1. The input part 100 includes, for example, an input interface. The data to be input is data to be used for training of the model NN. The data to be used for training may include, for example, verification data to be used for validation and the like in addition to teacher data related to input/output to/from the model NN. Further, the input part 100 may accept input of data such as a hyperparameter, an initial parameter, and the like.

The storage part 102 stores data required for the operation of the training device 1. For example, the data input via the input part 100 may be stored in the storage part 102. The storage part 102 is included in the training device 1 in FIG. 1, but at least a part of the storage part 102 may be mounted on an external storage, file server, or the like. In this case, the data may be input via the input part 100 at timing when the data is required.

The training part 104 executes training of the model NN. For example, the training part 104 forward propagates the data input via the input part 100 to the model NN, compares the output data and the teacher data to calculate an error, backward propagates the error, and appropriately updates the parameter constituting the model NN based on the information such as gradient.

The output part 106 outputs the data such as the parameter optimized by the training of the training part 104 to the external part or the storage part 102. As above, the output of the parameter of the like may be a concept including processing of storing the data in the storage part 102 of the training device 1 in addition to the output to the eternal part.

The model NN is a neural network model to be used in the NNP and, for example, a model which deduces interatomic interaction capable of acquiring a result of a quantum chemical calculation. For example, the model NN is a neural network model which outputs energy and force when information on an atom related to both the compound such as a molecule or crystal and information on an environment is input. In the NNP, for example, the force can be acquired by backward propagating the energy value. Hereinafter, the acquisition of the force may be executed by backward propagation.

In this description, the information on the atom to be input into the model NN to be used for the NNP is, for example, data containing the information on the type and the position of each atom or the like. In this description, the information on the atom is called information related to the atom in some cases. Examples of the information on the atom include information related to the type of the atom, the position of the atom, and so on. Examples of the information related to the position of the atom include information indicating the coordinates of the atom, information directly or indirectly indicating the relative positions between atoms, and so on. The information is expressed, for example, by the distance, the angle, the dihedral angle, or the like between atoms. By calculating the information on the distance between two atoms or the angle among three atoms from the information on the coordinates of atoms and using them for input into the NNP as the information on the positions of the atoms, it is possible to secure invariance for the rotation and translation and enhance the accuracy of the NNP. Further, the information related to the position of the atom may be information directly indicating the position or information calculated from the position. Further, information related to charge and information related to binding may be included in addition to the information related to the type of the atom and the position of the atom.

Besides, the model NN is configured as an arbitrary appropriate neural network model in order to execute deduction of the NNP. This configuration may be a configuration including, for example, a convolution layer and a fully connected layer, or may include a layer capable of inputting/outputting graph information or a convolution layer, but not limited to them, and may be formed as an appropriate neural network model. The model NN may be, for example, a model which receives input of information on a molecule as the graph information, or may be a model which receives input of information on a tree converted from a graph.

This model NN can be used for deduction of energy in binding of protein and a compound or the like or deduction of a reaction speed in a catalyst or the like, as a non-limiting example. In addition, the model NN can be used for deduction in processing using energy, force, and the like between compounds. More specifically, the model NN deduces the energy, force, and the like of a compound, protein, and the like, and uses a technique of, for example, an MD method using their values, and thereby can be used for deduction of energy in binding of protein and a compound, deduction of a reaction speed in a catalyst or the like.

FIG. 2 is a flowchart illustrating an example of the processing by the training device 1 according to this embodiment.

The training device 1 receives data required for training via the input part 100 (S100). This data may be stored in the storage part 102 as necessary. The data is data required for training such as the training data being the teacher data, the data on the hyperparameter for forming the neural network model and the data on the initial parameter, the verification data for executing the validation, and so on. The training data will explained later in detail.

Next, the training part 104 executes training using the input data (S102). The training part 104 first forms the neural network model (model NN) based on the hyperparameter and so on, receives input of the input data of the training data into the model NN, and executes forward propagation processing. The training part 104 executes backward propagation processing from an error between the data output from the model NN after completion of the forward propagation processing and the output data (teacher data) of the training data. Based on gradient information acquired by the backward propagation processing, the training part 104 updates the parameter in each layer of the model NN. The series of processing is repeated until end conditions are satisfied. The processing related to the training is executed by a general arbitrary appropriate machine learning technique.

After completion of the end conditions of the training and completion of update of the parameter, the output part 106 outputs the optimized parameter and ends the processing (S104). The inferring device acquires the parameter and the hyperparameter and forms the model NN, and thereby can constitute a deduction model to be used for the NNP.

Generally, for the training of the neural network model in the NNP, the data related to a compound such as a molecule or crystal is used. For these pieces of data, for example, data acquired using a simulation based on the physical law of the first-principles calculation such as the DFT (Density Functional Theory) or the like can be used. In this embodiment, energy and force are acquired by the DFT for the compound or the like, and a combination of this data and the input data is used as the training data. Note that in addition to the acquisition through the arithmetic operation of the DFT or the like, the training data may be acquired from a database or the like storing already calculated results.

Further, in this embodiment, the training device 1 uses the amounts of energy and force in two elements for the training of the model NN. These amounts are amounts according to the 2-body potential curve. For example, the training device 1 uses, as the training data, a data set according to the 2-body potential curve being information on interaction energy and force of two elements at various distances calculated by an arbitrary method.

This training data may be the one generated in advance by a training data generating device different from the training device 1. The training data generating device sets each of two atoms as an element whose data is to be acquired based on a simulation based on the physical law of the first-principles calculation or the like, for example, the DFT calculation, and acquires data required for training using the calculation by the simulation while changing the distance. In other words, the training data generating device sets two elements of the same type or different types, sets the distance between the two elements to various distances, acquires the energy and force at the various distances between the two elements using the first-principles calculation, and generates training data. The data set according to the 2-body potential curve is generated as above and used for training.

As another example, in the case where the 2-body potential is approximated as a function, elements and the potential at a distance based on the function or the like may be acquired. In this case, the accuracy may be inferior to the result of the arithmetic operation by the DFT or the like, but the result can be acquired more speedily. Therefore, the arithmetic processing time for the training data generation can also be reduced.

As the data related to the two elements, data which exists in the database and has been already known may be used as with the above-described data on the molecule or the like. On the other hand, data at an arbitrary distance can be generated by recalculation by the DFT or the like, so that the already known data can also be reinforced. Further, since the number of elements is limited, training data according to the 2-body potential curve may be acquired in advance by the DFT calculation for all of combinations of elements. Since the density of the acquisition of data can be enhanced as explained above, a neural network model which realizes deduction high in accuracy in interpolation and extrapolation can be formed.

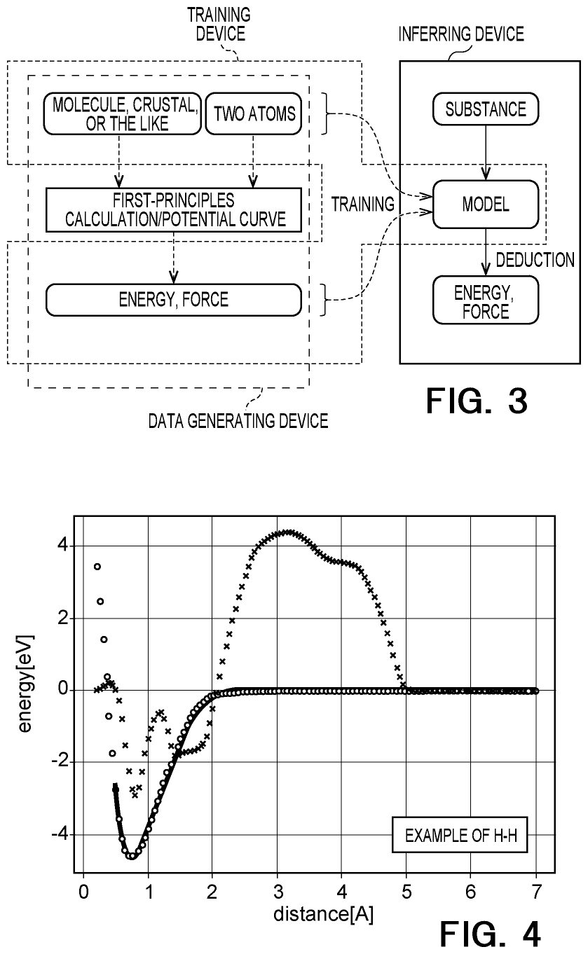

FIG. 3 is a diagram schematically illustrating the appearance of the data generation and training according to this embodiment. A broken line represents the data generating device, a dotted line represents the training device, and a solid line represents the inferring device. The data generating device may use, for example, the DFT as the first-principles calculation.

As software for executing the DFT, an arbitrary one such as VASP (registered trademark), Gaussian (registered trademark), or the like can be used with an arbitrary parameter. For example, as this software, the one that is the same as the software which has acquired the information on the molecule, crystal, or the like may be used with the same parameter. Besides, in the case of desiring to realize the deduction in the combination of predetermined software and a predetermined parameter, the data may be generated using the combination of the software and the parameter.

First of all, a general method will be explained. As explained above, generally the data generating device acquires the information on the energy and force using the first-principles calculation or the like from various states of the molecule, crystal, or the like. Using the information, the training device trains a model. The inferring device executes deduction using the trained model. The data to be used for the training is based on the molecule, crystal, or the like, and the data based on two atoms (data according to the 2-body potential curve) is not used for the training.

On the other hand, in this embodiment, the data generating device acquires the information on the energy and force using the first-principles calculation from various states of the molecule, crystal, or the like, and acquires the information on the interaction energy between two atoms according to the 2-body potential curve using the first-principles calculation from two atoms being an arbitrary combination of elements and the information on the distance between the two atoms. The training device executes training of the model using both of the information on the molecule, crystal, or the like and energy or the like and the information on the two atoms, energy, and the like. Besides, the inferring device executes deduction using this model. As explained above, for the training, the data based on the two atoms is used together with the information on the molecule, crystal, or the like.

Note that, as explained above, the data generating device does not have to be provided in the system, in which case the data existing in the database or the like may be used.

FIG. 4 is a graph illustrating an inference result of energy generated as above by the inferring device according to this embodiment and an inference result of energy by an inferring device (comparative example) trained without using the 2-body potential. This is a graph obtained by comparing the deduction results between the model NN trained by the training device 1 according to this embodiment and the comparative example in the case where two hydrogen elements exist as the two atoms.

A solid line represents energy between two atoms calculated under the condition of ωB97XD/6-31G(d) in the DFT. Plots expressed by round-shape-mark are obtained by NNP arithmetic operation using the model NN trained in this embodiment. Plots expressed by x-mark are obtained by NNP arithmetic operation using the model trained without using the 2-body potential as the comparative example.

As in this graph, it is found that the case of using the model NN trained by the training device 1 according to this embodiment is accurately approximated by the solid line. This shows that the deduction of the 2-body potential can be executed with high accuracy by the model NN.

On the other hand, it is found that in the comparative example, since the training using the data according to the 2-body potential curve is not executed, the extrapolation in the neural network model cannot be accurately executed.

A bond length of a hydrogen molecule is about 0.74 Å, and there is a local stable point near 1.4 Å in the comparative example. Therefore, there is such a problem in the MD simulation or the like that a hydrogen molecule having a bond length of about 1.4 Å that is originally unstable appears, and a result diverging from the reality is obtained. Comparatively, the deduction of the 2-body potential can be realized with higher accuracy according to this embodiment than that in the comparative example.

As explained above, according to this embodiment, it becomes possible to enhance the accuracy of not only the deduction of the 2-body potential but also the deduction of the energy of a compound such as a molecule or crystal, by using the data on a compound such as a molecule or crystal as the training data as being conventionally executed and using the data based on the 2-body potential, specifically, the data according to the 2-body potential curve as the training data in the training of the neural network model used in the NNP.

This is because of not only the deduction only in the 2-body potential but also the existence of energy and the like caused by the 2-body potential curve based on the 2-body potential function in the potential of the compound or the like. Therefore, optimization of the model NN so as to be able to deduce the interaction energy between two atoms and the like makes it possible for the 2-body potential function to appropriately include the deduction result based on the 2-body potential curve, and makes it possible to enhance the accuracy of the deduction of the energy and the like including a reaction path in another compound such as a molecule or crystal that is not two atoms.

Note that the example of acquiring the training data using the DFT as the first-principles calculation has been explained, but the acquisition of the training data does not exclude the use of another method such as the Hartree-Fock method.

Besides, the 2-body potential is explained in the above but may be restated as a potential between at least two atoms. More specifically, if a potential among atoms of three or more bodies can be appropriately calculated, the potential among the atoms of three or more bodies may be input as the data set to be used for training. In this case, the training of the model NN capable of further fitting with a potential function among many bodies can be realized.

### Second Embodiment

Though the inclusion of the data on the 2-body potential in the training data has been explained in the above embodiment, the embodiment in this disclosure is not limited to this.

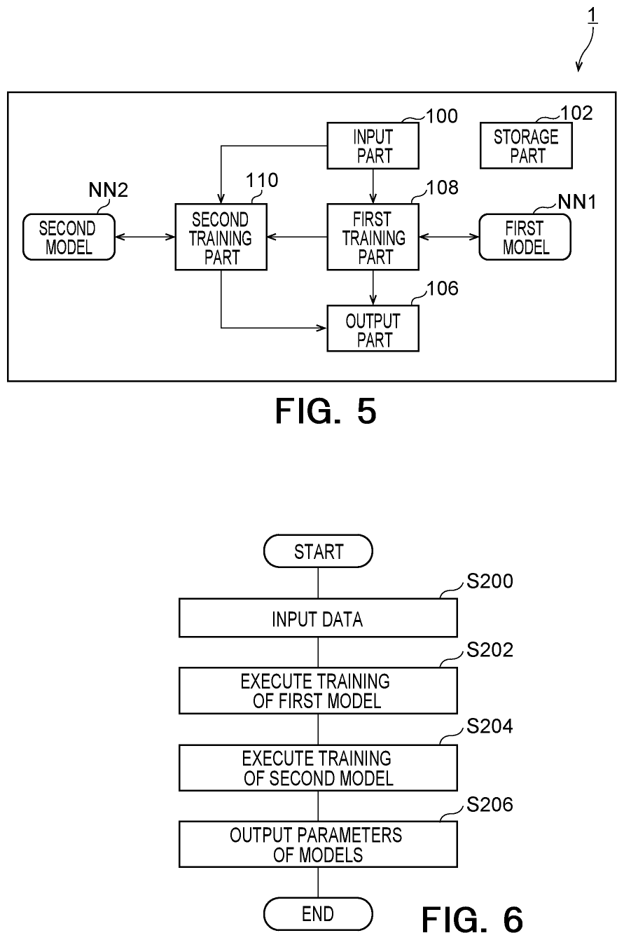

FIG. 5 is a block diagram illustrating an example of a training device according to a second embodiment. A training device 1 includes an input part 100, a storage part 102, a first training part 108, a second training part 110, and an output part 106. Components given the same codes as those in the first embodiment execute the same operations. The first training part 108 optimizes a first model NN1 by machine learning, and the second training part 110 optimizes a second model NN2 by machine learning.

The first model NN1 is a neural network model which outputs a 2-body potential when the types of and the distance between elements of two atoms are input.

The second model NN2 is a neural network model which outputs information on the energy or the like obtained by excluding energy related to the 2-body potential between constituting atoms when information on a molecule, crystal, or the like is input.

The first training part 108 trains the first model NN1 by an arbitrary machine learning technique appropriate for training the first model NN1 using the data set of the types of and the distance between elements of two atoms input via the input part 100 and energy according to a 2-body potential curve. The first training part 108 completes the training of the first model NN1 in advance before training of the second model NN2 is executed.

The second training part 110 executes the training of the second model NN2 in a state where the training of the first model NN1 is completed. Similarly to the first training part 108, the second model NN2 is trained by an arbitrary machine learning technique appropriate for training the second model NN2.

The first model NN1 and the second model NN2 are trained at different timing as explained above, but not limited to this. The training device 1 may train the first model NN1 and the second model NN2, for example, at the same timing. The training device 1 may, for example, input the data related to two atoms in the training data set into the first model NN1 and input the data related to a molecule, crystal, or the like in the training data set into the second model NN2. Then, the first model NN1 may be trained based on the output from the first model NN1 and the 2-body potential, and the second model NN2 may be trained based on a sum of the output from the first model NN1 and the output from the second model NN2 and the teacher data by the first-principles calculation or the like.

Note that the first model NN1 is not an essential configuration. For example, the first model NN1 may be replaced with a function which finds the 2-body potential based on the Lennard-Jones function, and another function or model may be used which can appropriately calculate the 2-body potential. This also applies to an inferring device 2 according to this embodiment.

FIG. 6 is a flowchart illustrating processing according to the second embodiment.

The training device 1 first acquires training data via the input part 100 (S200). The training data is data in which the state of two atoms (types of respective elements and the distance between the two atoms) required for the training of the first model NN1 and the 2-body potential (including energy and force) are associated, and the data in which the state of a molecule, crystal, or the like and the information including the energy and force are associated.

The first training part 108 executes training of the first model NN1 using the training data related to the 2-body potential (S202). The first model NN1 is a neural network model appropriate for inputting/outputting the information related to the 2-body potential. The first training part 108 trains the first model NN1 by a machine learning technique appropriate for training the first model NN1. The end conditions can also be arbitrarily decided. For example, the first training part 108 inputs the information on the two atoms into the first model NN1 and forward propagates it, and backward propagates an error between the output result and the information on the 2-body potential on the two atoms to update the parameter.

After completion of the training of the first model NN1, the second training part 110 executes training of the second model NN2 (S204). The second training part 110 trains the second model NN2 using the output data from the optimized first model NN1 and the training data on the molecule, crystal, or the like. As one example, the second model NN2 is trained so that a sum of the energy output from the first model NN1 and the energy output by inputting the information on the molecule, crystal, or the like into the second model NN2 becomes a value of the teacher data (an arithmetic result by the first-principles calculation or the like of a molecule, crystal, or the like).

For example, in the training of the second model NN2, the first training part 108 extracts information on the combination of two atoms from the information on the molecule or the like, and forward propagates the first model NN1 about the 2-body potential related to the two atoms for acquisition. The second training part 110 then inputs the information on the molecule or the like and forward propagates it to the second model NN2. The second training part 110 calculates an error with the teacher data by the first-principles calculation in consideration of the information on the energy or the like output from the first model NN1 based on the potential function in the information on the energy or the like output from the second model NN2, and backward propagates the error to execute the training of the second model NN2.

As one of the most simple examples, the second training part 110 executes the training of the second model NN2 using, as the teacher data, a difference between the amount of the energy or the like calculated by the first-principles calculation and the amount of the energy or the like between two atoms constituting a molecule or the like output from the first model NN1. In this case, the 2-body potential related to two atoms existing within a predetermined distance among all of the combinations of two atoms constituting a molecule or the like may be calculated by the first model NN1, a sum of the calculated energies or the like may be subtracted from the energy calculated by the first-principles calculation, and the resultant may be regarded as the teacher data. The predetermined distance can be a distance which can exert influence as the 2-body potential, for example, depending on the types of elements of the two atoms.

In addition to the above, data obtained by substituting the amount of the energy or the like between the two atoms constituting the molecule or the like output from the first model NN1 into an arbitrary potential function and excluding the energy caused by the 2-body potential may be used as the teacher data. Also in this case, the combination of two atoms existing within the predetermined distance may be extracted from the constitution of the molecule or the like and the 2-body potential may be calculated in the first model NN1 as in the above. The second training part 110 then executes training of the second model NN2 using the energy of the molecule of the like from which the influence of the 2-body potential is removed, as the teacher data, based on the potential function.

After completion of the training of the second model NN2, the information on the parameters or the like related to the first model NN1 and the second model NN2 is output via the output part 106, and the processing is ended (S206).

As explained above, the training device 1 trains the first model NN1 related to the 2-body potential and the second model NN2 related to the potential of the compound such as the molecule or crystal.

Note that the first model NN1 may be trained in advance by another training device. In this case, the training device 1 does not have to include the first training part 108 and may train the second model NN2 based on the output result from the first model NN1.

Besides, as explained above, the training device 1 may train the first model NN1 and the second model NN2 in parallel. In this case, the processing at S202 and the processing at S204 may be executed at the same timing.

Besides, the data related to the 2-body potential is not used in the training of the second model NN2 in the above example, but not limited to this. The second training part 110 may use the training data related to the 2-body potential in the training of the second model NN2. In this case, the second training part 110 may train the second model NN2 so that the energy (and force) becomes zero when the data related to the 2-body potential, namely, the data on two atoms is input.

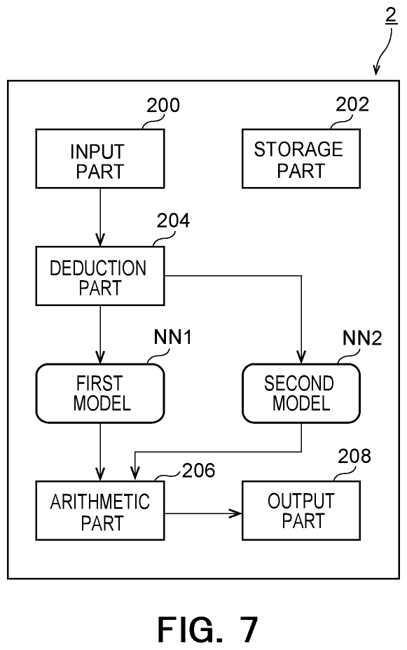

FIG. 7 is a block diagram illustrating an example of an inferring device according to this embodiment. The inferring device 2 includes an input part 200, a storage part 202, a deduction part 204, an arithmetic part 206, and an output part 208. The inferring device 2 infers and outputs a physical amount of energy or the like upon receiving input of information related to a compound such as a molecule or crystal.

The inferring device 2 receives input of data required for deduction via the input part 200. The input data may be temporarily stored, for example, in the storage part 202. A concrete operation of the input part 200 is the same as the operation of the input part 100 in the training device 1, and therefore its detailed explanation is omitted.

The storage part 202 stores data required for inference processing in the inferring device 2. The operation of the storage part 202 is also the same as the operation of the storage part 102 in the training device 1, and therefore its detailed explanation is omitted.

The deduction part 204 deduces the amount of energy or the like from the input information on the two atoms, molecule, or the like using the first model NN1 and the second model NN2 which have been trained in the training device 1. The deduction part 204 appropriately forward propagates the input information to the first model NN1 and the second model NN2 and performs deduction.

When information on two atoms is input, the deduction part 204 inputs the information on the two atoms into the first model NN1 to thereby execute inference processing of the 2-body potential, and outputs an inference result of the 2-body potential to the arithmetic part 206. In this case, the second model NN2 does not have to be used.

Information on three or more atoms, for example, information on a molecule, crystal, or the like is input, the deduction part 204 inputs the information on two atoms forming the 2-body potential function into the first model NN1 and inputs the information on the molecule, crystal, or the like into the second model NN2. As in the above-explained training time, the deduction part 204 extracts two atoms within the predetermined distance from the information on the molecule or the like, inputs the information on the two atoms into the first model NN1 and inputs the information on the molecule of the like itself into the second model NN2, and forward propagates them. The information output from each of the models is output to the arithmetic part 206.

Note that in the case of using the training data related to the 2-body potential in the second model NN2 as explained above in the training, the deduction part 204 may input the information on the extracted two atoms together with the information on the molecule or the like into the second model NN2.

The arithmetic part 206 acquires information on energy, force, and the like as a whole based on the information related to the 2-body potential output from the first model NN1 and the information on the potential in the molecule or the like output from the second model NN2. The arithmetic part 206 performs an arithmetic operation of the whole energy or the like using the same method as the method in consideration as the teacher data of the second model NN2 in the training.

For example, in the case of using the difference of the result of the 2-body potential from the result of the first-principles calculation as the teacher data of the second model NN2 in the training, the arithmetic part 206 calculates a sum of the output result of the first model NN1 and the output result of the second model NN2 and outputs the sum as the amount of the energy or the like.

For example, in the case of using the potential function that is not a simple sum in the training, the arithmetic part 206 performs an arithmetic operation of synthesizing the output from the first model NN1 and the output from the second model NN2 based on the potential function, and outputs a result of the arithmetic operation.

The output part 208 outputs the result of the arithmetic operation by the arithmetic part 206 as an inference result.

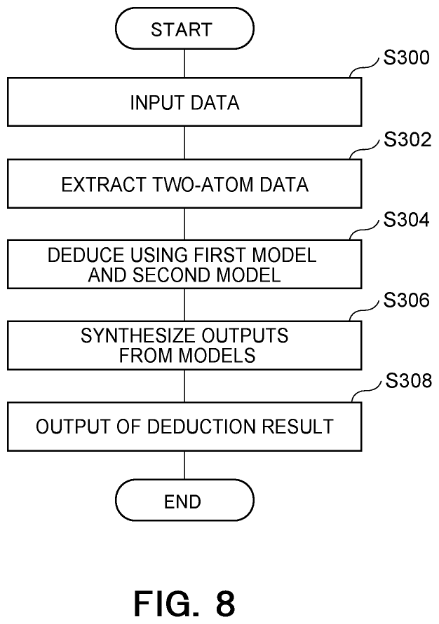

FIG. 8 is a flowchart illustrating processing by the inferring device 2 according to this embodiment.

The inferring device 2 first acquires data related to a molecular structure being a target of inference via the input part 200 (S300).

The deduction part 204 extracts data related to two atoms from the input data on the molecular structure or the like (S302). This processing may be processing of extracting all of combinations of two atoms or may be processing of extracting combinations of two atoms within a predetermined distance.

Next, the deduction part 204 forward propagates the data to the first model NN1 and the second model NN2 to thereby execute deduction of the potential (S304). The deduction part 204 inputs the extracted data related to the two atoms into the first model NN1 and inputs the data related to the molecule or the like into the second model NN2, and forward propagates the data.

Not limited to the above, the deduction part 204 inputs data according to a definition designed at the training of the second model NN2 into the second model NN2.

Upon acquisition of the outputs from the first model NN1 and the second model NN2, the arithmetic part 206 appropriately synthesizes the outputs (S306). As with the processing at Step S304, the arithmetic part 206 executes a synthetic arithmetic operation based on the method defined at the training.

The output part 208 then outputs the synthesis result by the arithmetic part 206 as the information on the energy, force, and the like (S308).

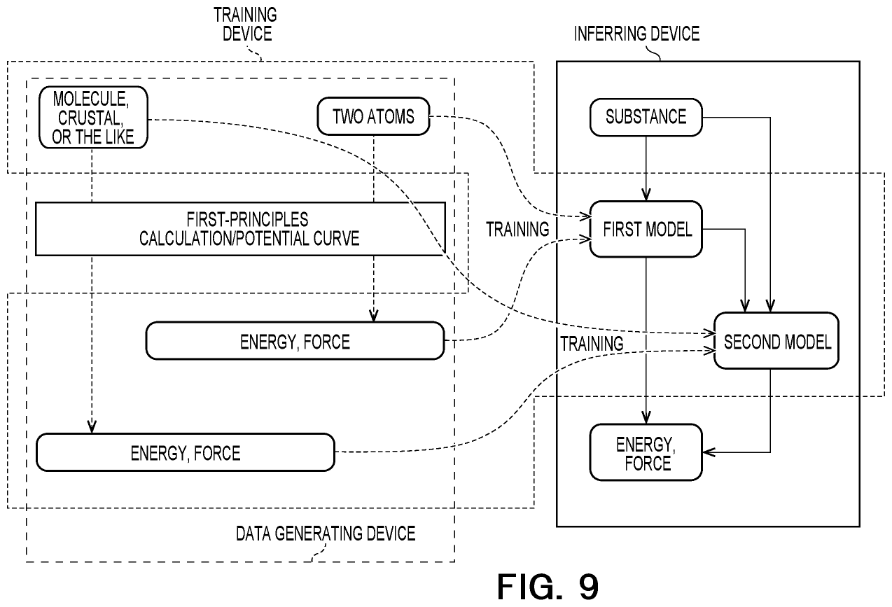

FIG. 9 is a diagram illustrating the flow of data in the data generating device, the training device, and the inferring device in this embodiment. As with FIG. 3, a broken line represents the data generating device, a dotted line represents the training device, and a solid line represents the inferring device.

The data generating device acquires the information on the energy, force, and the like based on the first-principles calculation or the potential curve from the information on the two atoms. Further, the data generating device acquires the information on the energy, force, and the like based on the first-principles calculation or the like from the information on the molecule, crystal, or the like.

The training device 1 trains the first model using the information on the two atoms and the information on the 2-body potential as the training data. Further, the training device 1 uses the information on the molecule, crystal, or the like and the output data of the first-principles calculation as the training data, and trains the inferring device 2 using this training data and the output from the first model.

The inferring device 2 deduces the information on energy or the like that can be expressed by the 2-body potential using the first model and the second model and energy or the like that cannot be expressed by the 2-body potential, from information related to a substance such as a molecule or crystal, synthesizes the deduction results to acquire the value of energy or the like, and outputs it.

As explained above, the training device according to this embodiment makes it possible to execute the training of the neural network model appropriately reflecting the 2-body potential, the structure of the molecule or the like, and the information on the energy or the like in a peripheral situation, based on the potential function. Further, the inferring device according to this embodiment can perform deduction while separating the structure of the molecule or the like, the energy in the environment, and the like into the 2-body potential and the potential related to the structure of the molecule or the like based on the potential function, and appropriately synthesize results of the deduction.

This deduction also makes it possible to appropriately acquire, for example, the potential or the like among three atoms or the like without undesirable aggregation. Further, optimization of the data at a short distance between two atoms as the training data makes it possible to acquire the potential in consideration of appropriate repulsive force, energy, or the like. For example, if the value of the 2-body potential is the teacher data for the model being the training target in the training, the learning as a whole may be affected by the amount of large energy or the like being a cause of repulsive force in this region in some cases, and the training and the deduction as a whole system may become unstable.

Training a portion (first model NN1) that can be expressed by the 2-body potential and a portion (second model NN2) that cannot be expressed by the 2-body potential as other models as in this embodiment makes it possible to remove factors which become unstable at the time of training and deduction.

Hereinafter, the data generation, training, and inference in this disclosure will be concluded.

The model NN in FIG. 1 and the first model NN1 and the second model NN2 in FIG. 5 which are the training targets are models each composed of the definition of a series of calculations and a set of parameters. These models may be arbitrary neural network models each capable of arbitrarily acquiring a solution, as not-limited examples. In the case where they are neural network models, the NNP in the inferring device is composed of these models.

At least the input to the model that deduces the 2-body potential, for example, the model NN, the first model NN1, (and may include the second model NN2) is an input of the type of an element of each atom in the two atoms and a sequence of sets of the three-dimensional coordinates of each atom. The training data is generated by first arbitrarily selecting types of elements constituting the two atoms and acquiring the amount of energy or the like by the potential curve or the first-principles calculation while changing the distance between the two atoms in this state. Then, the types of elements constituting the two atoms are arbitrarily changed, and the potentials between various elements and at various distances are acquired. For example, the amounts of the 2-body potential at the various distances may be acquired for all of the combinations of elements. This data set is described as a first data set.

At least the input to a model that deduces the amount that cannot be expressed by the 2-body potential, for example, the model NN and the second model NN2 is an input of the type of element of each of the atoms constituting the molecule or the like and a sequence of sets of the three-dimensional coordinates of the element, as the data related to the structure of the molecule, crystal, or the like. The training data is composed of a set of the structure information on the molecule or the like and the amount of energy or the like acquired using the first-principles calculation such as the DFT for the molecules or the like. This data set is described as a second data set.

In the first embodiment, the training device 1 executes training of the model NN using data obtained by integrating the first data set and the second data set as the training data.

In the second embodiment, the training device 1 trains the first model NN1 using the first data set as the training data and trains the second model NN2 using the input/output data of the first model NN1 and the second data set. The training device 1 trains the second model NN2 using, as the teacher data, for example, the energy or the like obtained by subtracting the sum of the output values obtained by inputting the combination of two atoms in the input molecule or the like into the first model NN1, from the energy or the like of the molecule or the like to be input in the second data set. Besides, an arithmetic operation based not on a simple sum but on a potential function may be executed.

As explained above, in this disclosure, explicitly using the data on the 2-body potential for training makes it possible to appropriately train one model in the first embodiment and each of two models in the second embodiment.

The trained models of above embodiments may be, for example, a concept that includes a model that has been trained as described and then distilled by a general method.

Some or all of each device (the inference device 1 or the training device 2) in the above embodiment may be configured in hardware, or information processing of software (program) executed by, for example, a CPU (Central Processing Unit), GPU (Graphics Processing Unit). In the case of the information processing of software, software that enables at least some of the functions of each device in the above embodiments may be stored in a non-volatile storage medium (non-volatile computer readable medium) such as CD-ROM (Compact Disc Read Only Memory) or USB (Universal Serial Bus) memory, and the information processing of software may be executed by loading the software into a computer. In addition, the software may also be downloaded through a communication network. Further, entire or a part of the software may be implemented in a circuit such as an ASIC (Application Specific Integrated Circuit) or FPGA (Field Programmable Gate Array), wherein the information processing of the software may be executed by hardware.

A storage medium to store the software may be a removable storage media such as an optical disk, or a fixed type storage medium such as a hard disk, or a memory. The storage medium may be provided inside the computer (a main storage device or an auxiliary storage device) or outside the computer.

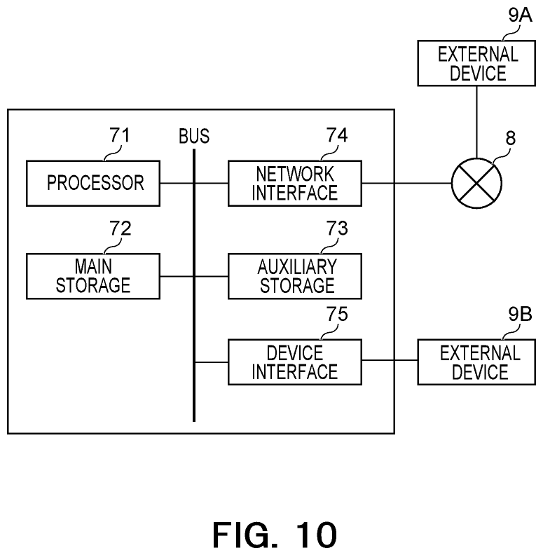

FIG. 10 is a block diagram illustrating an example of a hardware configuration of each device (the inference device 1 or the training device 2) in the above embodiments. As an example, each device may be implemented as a computer 7 provided with a processor 71, a main storage 72 (hereinafter a main storage device 72), an auxiliary storage 73 (hereinafter an auxiliary storage device), a network interface 74, and a device interface 75, which are connected via a bus 76.

The computer 7 of FIG. 10 is provided with each component one by one but may be provided with a plurality of the same components. Although one computer 7 is illustrated in FIG. 10, the software may be installed on a plurality of computers, and each of the plurality of computer may execute the same or a different part of the software processing. In this case, it may be in a form of distributed computing where each of the computers communicates with each of the computers through, for example, the network interface 74 to execute the processing. That is, each device (the inference device 1 or the training device 2) in the above embodiments may be configured as a system where one or more computers execute the instructions stored in one or more storages to enable functions. Each device may be configured such that the information transmitted from a terminal is processed by one or more computers provided on a cloud and results of the processing are transmitted to the terminal.

Various arithmetic operations of each device (the inference device 1 or the training device 2) in the above embodiments may be executed in parallel processing using one or more processors or using a plurality of computers over a network. The various arithmetic operations may be allocated to a plurality of arithmetic cores in the processor and executed in parallel processing. Some or all the processes, means, or the like of the present disclosure may be implemented by at least one of the processors or the storage devices provided on a cloud that can communicate with the computer 7 via a network. Thus, each device in the above embodiments may be in a form of parallel computing by one or more computers.

The processor 71 may be an electronic circuit (such as, for example, a processor, processing circuitry, processing circuitry, CPU, GPU, FPGA, or ASIC) that executes at least controlling the computer or arithmetic calculations. The processor 71 may also be, for example, a general-purpose processing circuit, a dedicated processing circuit designed to perform specific operations, or a semiconductor device which includes both the general-purpose processing circuit and the dedicated processing circuit. Further, the processor 71 may also include, for example, an optical circuit or an arithmetic function based on quantum computing.

The processor 71 may execute an arithmetic processing based on data and/or a software input from, for example, each device of the internal configuration of the computer 7, and may output an arithmetic result and a control signal, for example, to each device. The processor 71 may control each component of the computer 7 by executing, for example, an OS (Operating System), or an application of the computer 7.

Each device (the inference device 1 or the training device 2) in the above embodiments may be enabled by one or more processors 71. The processor 71 may refer to one or more electronic circuits located on one chip, or one or more electronic circuitries arranged on two or more chips or devices. In the case of a plurality of electronic circuitries are used, each electronic circuit may communicate by wired or wireless.

The main storage device 72 may store, for example, instructions to be executed by the processor 71 or various data, and the information stored in the main storage device 72 may be read out by the processor 71. The auxiliary storage device 73 is a storage device other than the main storage device 72. These storage devices shall mean any electronic component capable of storing electronic information and may be a semiconductor memory. The semiconductor memory may be either a volatile or non-volatile memory. The storage device for storing various data or the like in each device (the inference device 1 or the training device 2) in the above embodiments may be enabled by the main storage device 72 or the auxiliary storage device 73 or may be implemented by a built-in memory built into the processor 71. For example, the storage part 102 in the above embodiments may be implemented in the main storage device 72 or the auxiliary storage device 73.

In the case of each device (the inference device 1 or the training device 2) in the above embodiments is configured by at least one storage device (memory) and at least one of a plurality of processors connected/coupled to/with this at least one storage device, at least one of the plurality of processors may be connected to a single storage device. Or at least one of the plurality of storages may be connected to a single processor. Or each device may include a configuration where at least one of the plurality of processors is connected to at least one of the plurality of storage devices. Further, this configuration may be implemented by a storage device and a processor included in a plurality of computers. Moreover, each device may include a configuration where a storage device is integrated with a processor (for example, a cache memory including an L1 cache or an L2 cache).

The network interface 74 is an interface for connecting to a communication network 8 by wireless or wired. The network interface 74 may be an appropriate interface such as an interface compatible with existing communication standards. With the network interface 74, information may be exchanged with an external device 9A connected via the communication network 8. Note that the communication network 8 may be, for example, configured as WAN (Wide Area Network), LAN (Local Area Network), or PAN (Personal Area Network), or a combination of thereof, and may be such that information can be exchanged between the computer 7 and the external device 9A. The internet is an example of WAN, IEEE802.11 or Ethernet (registered trademark) is an example of LAN, and Bluetooth (registered trademark) or NFC (Near Field Communication) is an example of PAN.

The device interface 75 is an interface such as, for example, a USB that directly connects to the external device 9B.

The external device 9A is a device connected to the computer 7 via a network. The external device 9B is a device directly connected to the computer 7.

The external device 9A or the external device 9B may be, as an example, an input device. The input device is, for example, a device such as a camera, a microphone, a motion capture, at least one of various sensors, a keyboard, a mouse, or a touch panel, and gives the acquired information to the computer 7. Further, it may be a device including an input unit such as a personal computer, a tablet terminal, or a smartphone, which may have an input unit, a memory, and a processor.

The external device 9A or the external device 9B may be, as an example, an output device. The output device may be, for example, a display device such as, for example, an LCD (Liquid Crystal Display), or an organic EL (Electro Luminescence) panel, or a speaker which outputs audio. Moreover, it may be a device including an output unit such as, for example, a personal computer, a tablet terminal, or a smartphone, which may have an output unit, a memory, and a processor.

Further, the external device 9A or the external device 9B may be a storage device (memory). The external device 9A may be, for example, a network storage device, and the external device 9B may be, for example, an HDD storage.

Furthermore, the external device 9A or the external device 9B may be a device that has at least one function of the configuration element of each device (the inference device 1 or the training device 2) in the above embodiments. That is, the computer 7 may transmit a part of or all of processing results to the external device 9A or the external device 9B, or receive a part of or all of processing results from the external device 9A or the external device 9B.

In the present specification (including the claims), the representation (including similar expressions) of “at least one of a, b, and c” or “at least one of a, b, or c” includes any combinations of a, b, c, a-b, a-c, b-c, and a-b-c. It also covers combinations with multiple instances of any element such as, for example, a-a, a-b-b, or a-a-b-b-c-c. It further covers, for example, adding another element d beyond a, b, and/or c, such that a-b-c-d. In the present specification (including the claims), the expressions such as, for example, “data as input,” “using data,” “based on data,” “according to data,” or “in accordance with data” (including similar expressions) are used, unless otherwise specified, this includes cases where data itself is used, or the cases where data is processed in some ways (for example, noise added data, normalized data, feature quantities extracted from the data, or intermediate representation of the data) are used. When it is stated that some results can be obtained “by inputting data,” “by using data,” “based on data,” “according to data,” “in accordance with data” (including similar expressions), unless otherwise specified, this may include cases where the result is obtained based only on the data, and may also include cases where the result is obtained by being affected factors, conditions, and/or states, or the like by other data than the data. When it is stated that “output/outputting data” (including similar expressions), unless otherwise specified, this also includes cases where the data itself is used as output, or the cases where the data is processed in some ways (for example, the data added noise, the data normalized, feature quantity extracted from the data, or intermediate representation of the data) is used as the output.

In the present specification (including the claims), when the terms such as “connected (connection)” and “coupled (coupling)” are used, they are intended as non-limiting terms that include any of “direct connection/coupling,” “indirect connection/coupling,” “electrically connection/coupling,” “communicatively connection/coupling,” “operatively connection/coupling,” “physically connection/coupling,” or the like. The terms should be interpreted accordingly, depending on the context in which they are used, but any forms of connection/coupling that are not intentionally or naturally excluded should be construed as included in the terms and interpreted in a non-exclusive manner.

In the present specification (including the claims), when the expression such as “A configured to B,” this may include that a physically structure of A has a configuration that can execute operation B, as well as a permanent or a temporary setting/configuration of element A is configured/set to actually execute operation B. For example, when the element A is a general-purpose processor, the processor may have a hardware configuration capable of executing the operation B and may be configured to actually execute the operation B by setting the permanent or the temporary program (instructions). Moreover, when the element A is a dedicated processor, a dedicated arithmetic circuit, or the like, a circuit structure of the processor or the like may be implemented to actually execute the operation B, irrespective of whether or not control instructions and data are actually attached thereto.

In the present specification (including the claims), when a term referring to inclusion or possession (for example, “comprising/including,” “having,” or the like) is used, it is intended as an open-ended term, including the case of inclusion or possession an object other than the object indicated by the object of the term. If the object of these terms implying inclusion or possession is an expression that does not specify a quantity or suggests a singular number (an expression with a or an article), the expression should be construed as not being limited to a specific number.

In the present specification (including the claims), although when the expression such as “one or more,” “at least one,” or the like is used in some places, and the expression that does not specify a quantity or suggests a singular number (the expression with a or an article) is used elsewhere, it is not intended that this expression means “one.” In general, the expression that does not specify a quantity or suggests a singular number (the expression with a or an as article) should be interpreted as not necessarily limited to a specific number.

In the present specification, when it is stated that a particular configuration of an example results in a particular effect (advantage/result), unless there are some other reasons, it should be understood that the effect is also obtained for one or more other embodiments having the configuration. However, it should be understood that the presence or absence of such an effect generally depends on various factors, conditions, and/or states, etc., and that such an effect is not always achieved by the configuration. The effect is merely achieved by the configuration in the embodiments when various factors, conditions, and/or states, etc., are met, but the effect is not always obtained in the claimed invention that defines the configuration or a similar configuration.

In the present specification (including the claims), when the term such as “maximize/maximization” is used, this includes finding a global maximum value, finding an approximate value of the global maximum value, finding a local maximum value, and finding an approximate value of the local maximum value, should be interpreted as appropriate accordingly depending on the context in which the term is used. It also includes finding on the approximated value of these maximum values probabilistically or heuristically. Similarly, when the term such as “minimize” is used, this includes finding a global minimum value, finding an approximated value of the global minimum value, finding a local minimum value, and finding an approximated value of the local minimum value, and should be interpreted as appropriate accordingly depending on the context in which the term is used. It also includes finding the approximated value of these minimum values probabilistically or heuristically. Similarly, when the term such as “optimize/optimization” is used, this includes finding a global optimum value, finding an approximated value of the global optimum value, finding a local optimum value, and finding an approximated value of the local optimum value, and should be interpreted as appropriate accordingly depending on the context in which the term is used. It also includes finding the approximated value of these optimal values probabilistically or heuristically.

In the present specification (including claims), when a plurality of hardware performs a predetermined process, the respective hardware may cooperate to perform the predetermined process, or some hardware may perform all the predetermined process. Further, a part of the hardware may perform a part of the predetermined process, and the other hardware may perform the rest of the predetermined process. In the present specification (including claims), when an expression (including similar expressions) such as “one or more hardware perform a first process and the one or more hardware perform a second process,” or the like, is used, the hardware that perform the first process and the hardware that perform the second process may be the same hardware, or may be the different hardware. That is: the hardware that perform the first process and the hardware that perform the second process may be included in the one or more hardware. Note that, the hardware may include an electronic circuit, a device including the electronic circuit, or the like.

While certain embodiments of the present disclosure have been described in detail above, the present disclosure is not limited to the individual embodiments described above. Various additions, changes, substitutions, partial deletions, etc. are possible to the extent that they do not deviate from the conceptual idea and purpose of the present disclosure derived from the contents specified in the claims and their equivalents. For example, when numerical values or mathematical formulas are used in the description in the above-described embodiments, they are shown for illustrative purposes only and do not limit the scope of the present disclosure. Further, the order of each operation shown in the embodiments is also an example, and does not limit the scope of the present disclosure.

1. An inferring device comprising:
one or more memories; and
one or more processors configured to:
input information on each atom in an atomic system into a second model to infer a difference between energy based on a first-principles calculation corresponding to the atomic system and energy of an interatomic potential function corresponding to the atomic system.

2. The inferring device according to claim 1, wherein
the information on each atom includes at least information on three-dimensional coordinates of an atom or information on relative positions between atoms.

3. The inferring device according to claim 1, wherein
the one or more processors are configured to output an inferred value of the energy based on the first-principles calculation by using the interatomic potential function corresponding to the atomic system and an inferred value of the difference.

4. The inferring device according to claim 3, wherein
the one or more processors are configured to output force corresponding to the atomic system from the inferred value of the energy based on the first-principles calculation.

5. The inferring device according to claim 3, wherein
the inferred value of the energy based on the first-principles calculation is a sum of the energy of the interatomic potential function corresponding to the atomic system and the inferred value of the difference.

6. The inferring device according to claim 3, wherein
the one or more processors are configured to calculate the energy of the interatomic potential function corresponding to the atomic system from the information on each atom in the atomic system.

7. The inferring device according to claim 1, wherein
the one or more processors are configured to:
input the information on each atom in the atomic system into a first model to infer the energy of the interatomic potential function corresponding to the atomic system; and
input the information on each atom in the atomic system into the second model to infer a difference between the energy based on the first-principles calculation corresponding to the atomic system and the energy inferred using the first model, as inference processing of the difference.

8. The inferring device according to claim 7, wherein
the one or more processors are configured to output an inferred value of the energy based on the first-principles calculation by using the energy inferred using the first model and the difference inferred using the second model.

9. The inferring device according to claim 7, wherein
the one or more processors are configured to extract a combination of two atoms for each atom in the atomic system, and input the combination into the first model.

10. The inferring device according to claim 1, wherein
calculation based on the first-principles calculation is performed by DFT.

11. The inferring device according to claim 1, wherein
the interatomic potential function is Lennard Jones Potential.

12. The inferring device according to claim 1, wherein
the interatomic potential function is a 2-body potential function.

13. The inferring device according to claim 1, wherein
the interatomic potential function is an interatomic potential function of three or more bodies.

14. An inferring method comprising:
input, by one or more processors, information on each atom in an atomic system into a second model to infer a difference between energy based on a first-principles calculation corresponding to the atomic system and energy of an interatomic potential function corresponding to the atomic system.

15. The method according to claim 14, further comprising:
outputting, by the one or more processors, an inferred value of the energy based on the first-principles calculation by using the interatomic potential function corresponding to the atomic system and an inferred value of the difference.

16. The method according to claim 15, wherein
the inferred value of the energy based on the first-principles calculation is a sum of the energy of the interatomic potential function corresponding to the atomic system and the inferred value of the difference.

17. The method according to claim 14, further comprising:
inputting, by the one or more processors, the information on each atom in the atomic system into a first model to infer the energy of the interatomic potential function corresponding to the atomic system;
wherein the inputting the information on the each atom in the atomic system into the second model includes inferring a difference between the energy based on the first-principles calculation corresponding to the atomic system and the energy inferred using the first model, as inference processing of the difference.

18. The method according to claim 17, further comprising:
outputting, by the one or more processors, an inferred value of the energy based on the first-principles calculation by using the energy inferred using the first model and the difference inferred using the second model.

19. A training device comprising:
one or more memories; and
one or more processors configured to:
train a second model which, when receiving input of information on each atom in an atomic system, outputs an inferred value of a difference between energy based on a first-principles calculation corresponding to the atomic system and energy of an interatomic potential function corresponding to the atomic system.

20. The training device according to claim 19, wherein:
the one or more processors are configured to train a first model which, when receiving input of the information on each atom in the atomic system, outputs an inferred value of the energy of the interatomic potential function corresponding to the atomic system; and
the second model is trained to output an inferred value of a difference between the energy based on the first-principles calculation corresponding to the atomic system and energy inferred using the first model, as the inferred value of the difference
